(19) United States
(12) Patent Application Publication
(10) Pub. No.: US 2008/0228132 A1
(43) Pub. Date: Sep. 18, 2008

(54) Valve for Transcutaneous Access to Existing Blood Vessel or Fistula

(76) Inventor: John Langenbach, St. Louis, MO (US)

Correspondence Address: LEWIS, RICE & FINGERSH, LC;ATTN: BOX IP DEPT., 500 NORTH BROADWAY, SUITE 2000, ST LOUIS, MO, 63102, US

(21) Appl. No.: 11/994,734

(22) PCT Filed: Jul. 8, 2005
(86) PCT No.: PCT/US05/24224

§ 371 Date: Jan. 4, 2008

(51) Int. Cl. A61M 39/06

(52) U.S. Cl. 604/30

(57) ABSTRACT

A valve for facilitating the introduction of fluids into or withdrawal of fluids from the vascular system of an animal, including a human animal, which connects to a natural or artificial blood vessel to allow access to blood in the vascular system without the need to additionally puncture the skin with needles. The valve will often be connected to a pre-installed fistula interconnecting natural blood vessels.

### BACKGROUND

1. Field of the Invention

This disclosure relates to the field of transcutaneous devices and more particularly to a valve which may be implanted into a natural or artificial blood vessel (fistula) to facilitate access to the vascular system without need for needle penetration of the blood vessel or skin.

2. Background of the Invention

Human skin is designed to protect the contents of the human body, but often in medical procedures, access to those contents is required. While it may be medical necessity, breaking the skin is painful to a patient. It can also result in infection. While most people only have to bear medical needles and incisions periodically, some are forced to live with them on an almost daily basis.

Those whose kidneys have failed may still lead relatively normal lives by undergoing hemodialysis, perhaps three or four times per week. The procedure traditionally involved inserting two catheters through the skin and into accessible blood vessels using needles. The first catheter is used to withdraw blood from the blood vessel while the other is used to reintroduce the same blood into the circulatory system after it has been cleansed by external machinery.

Natural blood vessels are not built to sustain repeated penetrations by needles without collapsing. Typically, a patient who requires hemodialysis on a regular basis, therefore, undergoes a surgical procedure in which an artificial blood vessel (a graft or fistula) is grafted onto an artery and a vein in the patient's arm to serve as a shunt allowing access to the blood and vascular system without risking blood vessel collapse from repeated trauma.

Being formed from a polymer, the fistula can withstand repeated penetrations by needles. Even so, the fistula still lies beneath the skin, and with dialysis the needles must penetrate the skin before entering the artificial blood vessel. The penetrations can be painful and may produce infections. Further, they can be damaging to the skin over a lengthy period of time.

To try and deal with this problem, U.S. Pat. No. 6,726,711 provided for an artificial blood vessel including two transcutaneous ports. While this is an effective solution, the ports require a significant amount of subcutaneous space and are not intended to be used in conjunction with a natural blood vessel due to their size. Further, the port is offset from the principle flow path of the blood vessel requiring additional space and significant alteration of blood flow patterns through the vessel. The port also requires significant healing time before becoming strongly attached to the skin. Therefore, it would be desirable in the art to have a valve system which can be attached directly to an existing and in-place natural or artificial blood vessel and which does not require as significant subcutaneous space, and that can have improved attachment strength to human skin.

### SUMMARY

The present invention provides for a valve for facilitating access to a blood vessel, particularly to a previously implanted artificial blood vessel or fistula connecting the natural blood vessels. The valve will generally either help with the withdrawal of blood from the blood vessel, or with returning blood into the blood vessel, or providing medications or substances into the vascular system.

Described herein, amongst other things, there is a valve comprising: a connecting member for attaching the valve to a fistula; a grommet, the grommet designed to be located on the exterior of the skin of a living animal, the grommet being connected to the connecting member; an access passage, the access passage configured to project through the skin of the living animal, allowing fluid communication from a lumen in the fistula through the connecting member, the grommet, and to the exterior of the skin; a valve element which normally bears against a sealing surface in the access passage to isolate the access passage from the lumen, but is displaceable away from the sealing surface such that the valve element and sealing surface are separated; thus enabling the access passage to be in fluid communication with the lumen.

In an embodiment of the valve, the sealing surface lies in a spherical envelope, and the valve element is spherical. The valve element may be biased toward the sealing surface such as, but not limited by having a valve seat formed from a ferrous metal, with the sealing surface being on the valve seat and the passage extending through the valve seat; and wherein the valve element is magnetized or by having the valve element biased via a spring.

In an embodiment, the valve further comprises a cage which encloses a cavity which communicates with a lumen of the fistula; wherein the passage opens into the cavity, and the valve element is loosely received in the cavity.

In an embodiment, the valve element is a piece of deformable material.

In an embodiment, the grommet includes a plurality of locking pins, the locking pins allowing the grommet to be attached to a locking ring. The attachment may occur in a manner such that the skin of the living animal is held between the locking ring and the grommet.

In an embodiment the living animal is a human being and the fistula may be a preinstalled fistula located in an arm of the human being.

### BRIEF DESCRIPTION OF THE FIGURES

FIG. 1 illustrates a human arm having valves implanted therein. The valves are connected to a previously installed fistula connected between two natural blood vessels;

FIG. 2 is a sectional view of an embodiment of a valve;

FIG. 3 is a sectional view taken along line 3-3 of FIG. 2.

FIG. 4 is an exploded sectional view of the embodiment of FIG. 2.

FIG. 5 is a sectional view of a second embodiment of a valve.

FIG. 6 is a sectional view of third embodiment of a valve.

### DESCRIPTION OF PREFERRED EMBODIMENT(S)

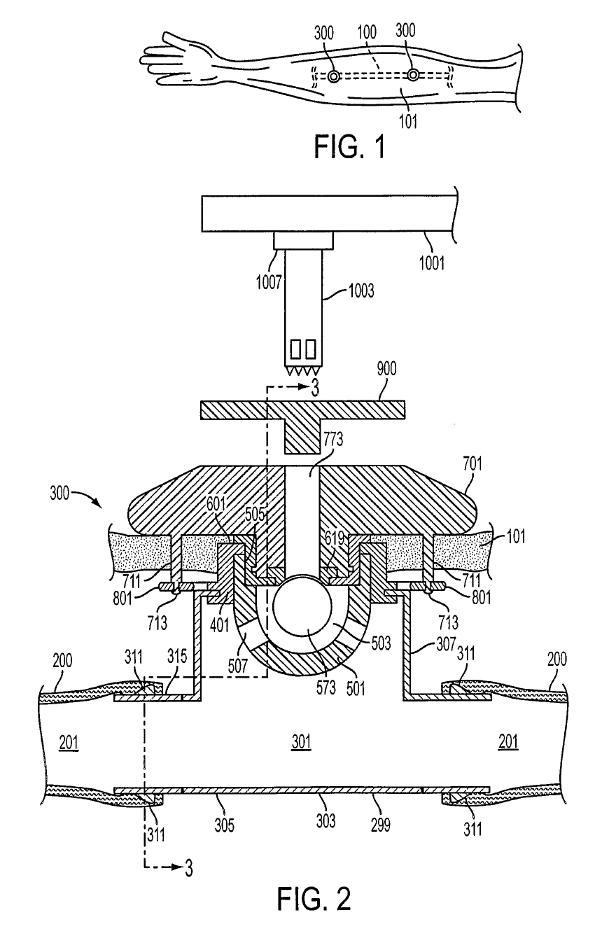

Referring to FIG. 1. An individual who requires hemodialysis or some other medical treatment that necessitates introducing fluids into and/or withdrawing fluids from the individual's vascular system or other internal organs or structures may have that introduction performed via a fistula (100) which has been placed in their body. The fistula (100) in the case of vascular access for hemodialysis will generally be located in the arm but that is by no means required. The fistula (100) may either be a natural blood vessel of the patient moved to perform this task, or, in most cases, an artificial blood vessel implanted to facilitate access to blood without the same risk of damage. For those whose need of hemodialysis is essentially permanent, the installation of such an artificial blood vessel is a virtual necessity to provide for a ready location for access by hemodialysis machines without as much risk of vascular degeneration. The fistula (100) will usually be located beneath the skin (101) of that individual and may represent a shunt between a natural artery and a natural vein as shown in FIG. 1. While the valves of the current disclosure may be attached to an existing fistula, a natural blood vessel or a grafted natural blood vessel depending on embodiment, the remainder of this disclosure will presume the valve is being attached to an existing fistula to simplify discussion.

Turning now to FIGS. 2 through 6, the fistula (100) comprises a tubular portion (200) through which blood flows. This tubular portion (200) will generally be formed from a substance that is flexible and compatible with the body in the sense that it is not toxic and the natural vessels to which it is connected will graft onto it. One exemplary suitable material is a polymer sold under the trademark Gore-Tex by W. L. Gore & Associates, Inc., of Newark, Del. Throughout its length, the tubular portion (200) has a hollow interior or lumen (201) providing for free flow of blood through the fistula (100) generally in a predetermined direction.

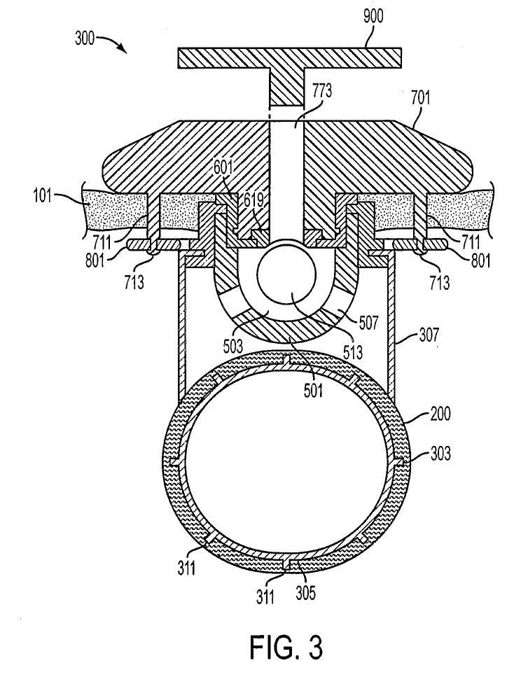

The valve (300) is designed to interlink to the existing fistula (100) to provide for an external access, outside the skin, to the vascular system currently accessed through the fistula (100). This connection is performed via a connecting member (303) which is designed to be permanently connected to the fistula (100) in a relatively minor surgical procedure. In a first embodiment shown in FIGS. 2 through 4, the valve (300) has towards its bottom end a generally “T” shaped protuberance (299) to act as the connecting member (303). The “T” shaped protuberance (299) encloses a hollow interior (301) which forms a part of the lumen (201) when connected. It is preferred that the lower portion of the protuberance (299), which is called the cross channel (305), have inner and outer diameters similar to the diameters of the unobstructed fistula (100) into which the valve (300) is to be inserted so that the resultant fluid pathway is of similar dimensions to the unmodified fistula (100). The upper neck (307) of the protuberance may comprise a linear extension, or may have an angled rise leading to the top of the “T”. The embodiment of FIGS. 2 and 3 shows a linear “T,” but it is important to recognize that a true “T” shape is not required and in an alternate embodiment, the protuberance (299) may comprise a “Y,” triangular, or related shape.

The cross channel (305) of the “T” protuberance (299) is designed to be permanently mounted to the fistula (100). In the embodiment of FIGS. 2 through 4, the fistula (100) is split perpendicular to its major axis and each end is attached to one end of the cross channel (305). To facilitate the connection, the cross channel (305) may include an outer segment (315) purposefully designed to be attached to the fistula (100). To facilitate such attachment, the outer segment (315) of the cross channel (305) may be constructed of materials designed to connect to the fistula (100) or may include attachment fins or barbs (311) designed to latch to the Gore-Tex of the fistula (100). If grafting to a natural vessel, the outer segment (315) may comprise Gore-Tex to facilitate grafting to the vessel in the same way that the fistula (100) is grafted to existing blood vessels.

It should be recognized, that the valve (300) is designed to be placed in line with the major flow channel of the fistula (100). In this embodiment, therefore, the fistula (100) is effectively cut perpendicular to its major axis to produce two ends which are then grafted onto the ends of the cross channel (305). In this arrangement, the valve (300) mechanism sits above the fistula (100) and there is no need for a special pocket or other blood-collecting area to feed the valve (300). The flow of blood through the fistula (100) will direct the blood into the valve (300). The cross channel (305) therefore is within (or more particularly forms) a small portion of the fistula (100) extending the lumen (201).

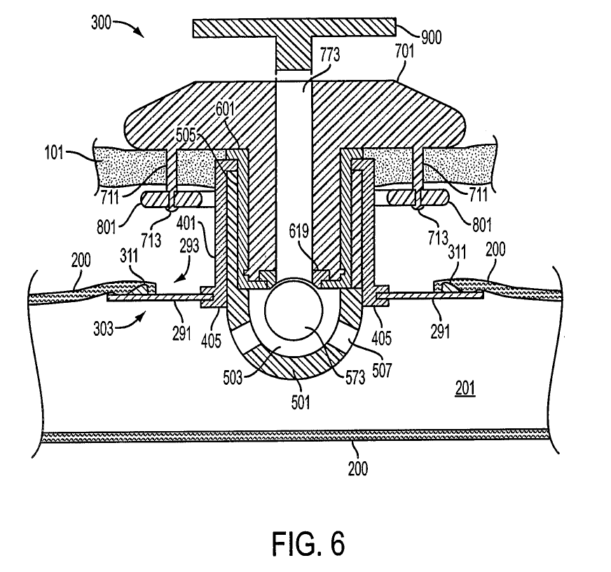

FIG. 6 shows an alternative embodiment whereby the valve (300) is grafted more directly to a fistula (100). In this case, the protuberance is effectively eliminated from the connecting member (303). Instead, the connecting member (303) comprises two small attachment tabs (291) which may be constructed of Gore-Tex. The tabs (291) are securely mounted to the valve (300). In this embodiment, the fistula (100) is directly accessed from above and is not split into two portions by the presence of the valve (300). Instead, a hole (293) is cut in the upper surface of the fistula (100) allowing the lumen (201) to be directly placed in contact with the valve (300) without supplementation.

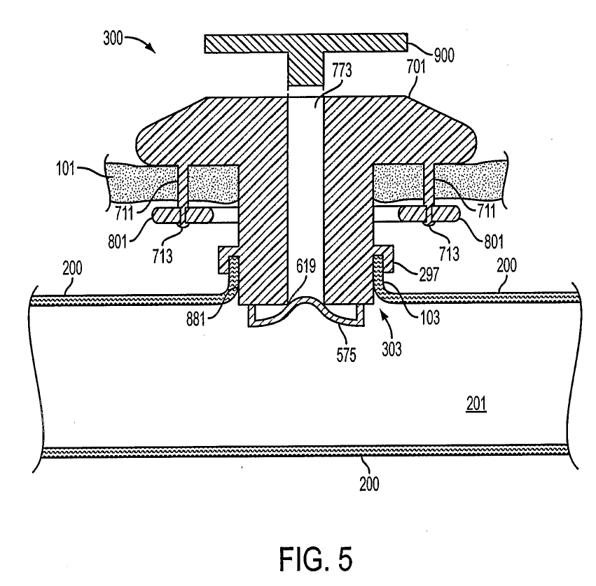

FIG. 5 shows a still further embodiment of connection. In FIG. 5, no or only a small portion of the fistula (100) is removed. Instead, the upper surface of the fistula (100) is simply split. The loosened “flaps” (103) of fistula (100) are then securely clamped in clamps (297) (which comprise the connecting member (303)) to hold the valve (300) to the fistula (100) and seal the valve (300) against the opening in the fistula (100). All these FIGS. merely show exemplary embodiments of in-line attachment to the fistula (100). In other embodiments, the connecting member (303) may utilize components of these depicted embodiments to provide for hybrid connections utilizing elements of some or all the described embodiments. For example, the connecting member (303) of FIG. 5 or 6 may replace the cross channel (305) of FIG. 1, but the upper neck (307) may still be present. Alternatively, other types of connecting members (303) may be used as would be understood by one of ordinary skill in the art.

For the most part, the valve (300) is formed from components that are molded from a polymer that is nontoxic to the individual's body. The polymer from which the components of the valve (300) are formed is relatively inflexible so that the valve (300) will maintain its shape, however different components may be manufactured of different materials as necessary.

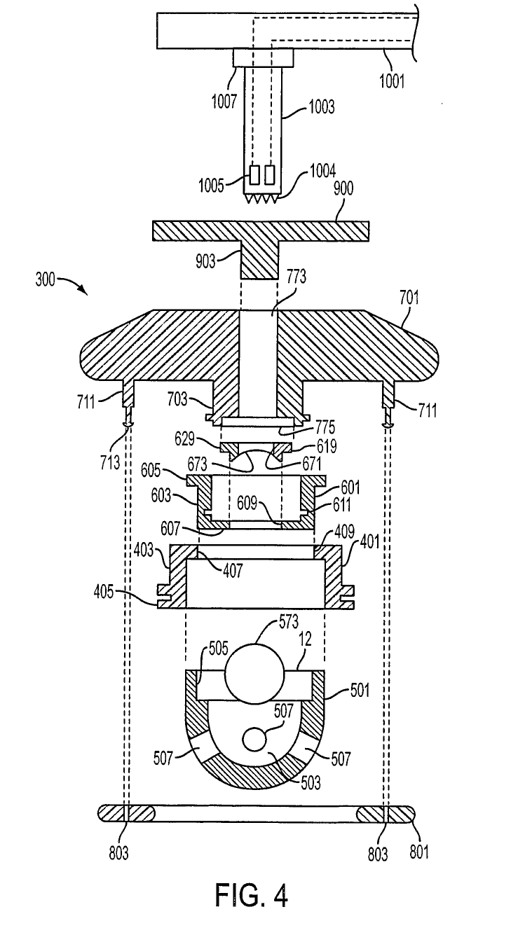

In the embodiments of FIGS. 2 through 4 and FIG. 6, the valve is composed of a number of discrete components which are assembled together to form the valve unit. The components are generally similar between the embodiments but are differently sized between embodiments. Among the molded components is a rim (401) having a side wall (403) provided at is lower end with an attachment mechanism (405). The attachment mechanism (405) is designed to attach to the connecting member (303) whether by attaching to tabs (291), the protuberance (299) or directly by clamps (297). The rim (401) also has an upper wall provided with an opening (407). The side wall (403) of the rim (401) interconnects with the connecting member (303) in a manner appropriate for the specific type of connecting member (303). In the embodiment of FIG. 4, the wall of the protuberance (299) interlocks with and is bonded to the rim (401) by the connector (405) in that a portion of the upper neck (307) extends into the gap between the two segments of the connector (405).

In addition, the valve (300) includes a cage (501) which fits snugly into the side wall (403) of the rim (401) from beneath and provides a cavity (503) and a counter bore (505) above the cavity (503). The cage (501) extends downward into the upper neck (307) of the protuberance (299) or may be similarly arranged to sit above the fistula (100) in a similar upper neck (307), or may alternately be arranged to extend into the lumen of the fistula (100) 9, as shown in FIGS. 5 and 6. In the event the cage (501) is above the fistula (100), there will be at least a minimum of clearance between the side wall of the cage (501) and the side wall of the upper neck (307). The cavity (503) communicates with the cross channel (305), and/or with the lumen of the fistula (100) through several apertures (507) in its wall. The apertures (507) are preferably located toward the lower end of the cage (501) and are preferably arranged so as to have at least a somewhat downward projection from the side of the cage (501) if the cage (501) is above the lumen (201). In an embodiment, the apertures (507) are arranged to specifically be on the bottom surface of the cage (501). In a still further embodiment, the cage (501) comprises a mesh or similar structure comprising mostly apertures but with a solid form. As should be apparent from the FIGS., the cage (501) provides a relatively open access to the apertures (507) from the lumen (201) of the fistula (100).

The rim (401), in its circular opening (407), receives a liner (601) having a side wall (603) that extends into the counter bore (505) of the cage (501). The liner (601) at its upper end has a flange (605) which overlies the upper wall (409) of the rim (401) and at its lower end a bottom wall (607) is provided with an aperture (609) that leads into the cavity (503) of the cage (501). Within its side wall (603) the liner (601) is provided with attachments (611). The liner (601) will generally be bonded or otherwise secured to the rim (401) along its flange (603) and to the cage (501) along its side wall. In an alternative embodiment, the cage (501), rim (401), and liner (601) are molded as a single monolithic piece.

The aperture (609) holds a valve seat (619) which is preferably formed from stainless steel or other substance capable of being attracted by a magnet. The seat (614) extends into the aperture (609) of the liner (601) and has a flange (629) which overlies the bottom wall (603) of the liner (601). The seat (619) also contains an aperture in the form of a through bore (671) which extends through it and opens into the cavity (503) of the cage (501). At its bottom edge, the valve seat (629) may include a contoured sealing surface (673).

Externally of the skin (101), the valve (300) has a grommet (701) provided on its underside with a boss (703) that passes through a prepared opening in the skin (101) and fits adjacent the side wall (603) of the liner (601). Extending through the grommet (701) and the boss (703) is an access bore (773) which opens into a shallow counter bore (775) at its lower end. Whereas the boss (701) slides into the liner (601) with a slight clearance, counter bore (775) at its lower end receives the flange (629) of the valve seat (619) again with a slight clearance. The valve seat (619) is captured between the bottom wall (607) of the liner (601) and the boss (703) of the grommet (701), so the valve seat (619) cannot be readily displaced. When the grommet (701) is so positioned, the access bore (773) in the grommet (701) aligns with and opens into the through bore (673) in the valve seat (601), so the two bores (773) and (673) form an access passage.

In the embodiment of FIG. 5, the valve (300) is constructed of far fewer pieces but still has a similar resultant design. In this case, the main body (881) of the valve is formed as a monolithic piece with the valve seat (619) molded therein during the formation of the main body (881). Either construction, or, in fact, construction using alternative designs, may be used in the various embodiments to provide for the desired resulting structure.

To attach the valve (300) to the skin (101), the grommet (701), on its underside, has several anchor pins (711). The anchor pins (711) will generally be arranged circumferentially about the bottom side of the grommet (701) at predetermined intervals. Each anchor comprises a thin post tipped with an expansion head or barb (713) which will extend from the bottom surface of the grommet (701) through the skin (101). The anchor pin's (711) barbs (713) are designed to lock with a locking ring (801) or similar structure under the skin (101) allowing the pin (711) to pass a first direction through a hole (803) in the locking ring (801), but not allowing the pin (711) to pass back out of the hole (803).

So as to hold the valve (300) to the skin (101), the locking ring (801) will generally be placed under the skin (101) during installation of the valve (300). The skin (101) will then either be sewn back together over the locking ring (801), or will simply be returned to a position over the ring (801) depending on procedure. The anchor pins (711) will be extended through the skin (101) and into mating holes (803) in the ring (801) where they lock in place by having the barbs (713) extend and grip the underside of the ring (801). The skin (101) grows around the anchor pins (711) to further retain the valve in position in the skin (101). The connection is strong as the skin (101) has been allowed to remain and continue to grow through the space between the locking ring (801) and the grommet (701). In lieu of being formed from a polymer, the grommet (701) may be formed from a nontoxic metal as may the ring (801).

In the embodiments depicted in FIGS. 2 through 4 and 6, the cavity (503) within the cage (501) contains a ball (573) or other generally rounded or spherical object having essentially the same radius as the radius of the sealing surface (673) on the valve seat (619). This acts as a valve element in the valve (300). Moreover, the ball (573) is preferably formed from a substance that is permanently magnetized so that the magnetic force attracts it to the valve seat (619). The ball (573) therefore forms the valve element which, owing to the magnetic attraction, normally remains against the sealing surface (673) on the valve seat (601), thus blocking the access bore (773) so that blood which flows through the cage (501) will not escape into the access bore (773) as the ball (573) is in the way. Further, the connection between the ball (573) and the valve seat (601) has sufficient tension to resist the force of blood flowing past the ball (573), under most circumstances, and will remain in place. The ball (573) may also be biased against the valve seat (601) with a spring located between it and the bottom surface of the cage (501) or any other biasing member known to those of ordinary skill in the art.

In the embodiment of FIG. 5, the ball (573) is replaced with an alternative valve element to seal the access passage. In this embodiment the valve element comprises a piece of a deformable material (575), such as spring steel, and a biasing member (if necessary) for returning the deformable material (575) to a first-predetermined arrangement whereby it seats against the valve seat (619). This design is preferred in some embodiments where the cage (501) would sit in the lumen (201) to provide for less resistance to blood flow and decreased surface effects. The design further provides for lessened surface area contact with the blood compared to the ball (573) which can provide a more solid seat. In particular, blood rushing through the lumen (201) and impacting the ball (573) could dislodge the ball (573) from the valve seat (619) if the magnetic attraction or other biasing is insufficiently strong or the blood flow is sufficiently powerful. The design of FIG. 5 decreases this possibility by decreasing the surface area of the valve element (the deformable material (575)) in contact with the blood flow. This makes the valve harder to open without intentional pressure applied from within the access passage. In this embodiment, magnetic attraction between the valve element (the deformable material (575)) and the valve seat may still be utilized, however, the valve seat in this embodiment is preferably permanently magnetized.

The valve will generally have a removable closure (900) which fits over the top of the grommet (701) and is provided with a cylindrical plug (903) that fits into the access bore (773). When in place, the closure (900) inhibits contaminants from entering the bore (773). The closure (900) is easily removed from the grommet (701) to expose the access bore (773) when access is needed by simply disengaging it.

Introduction or withdrawal of fluids will generally be through a cannula (1001) such as those currently used in hemodialysis which has a connector prong (1003) small enough in diameter to fit into the access bore (773) of the grommet (701). At its end, the connector prong (1003) has openings (1005) through which the hollow interior of the cannula (1001) is exposed. In addition, the cannula (1001) preferably has a shoulder (1007) which is too large to fit into the access bore (773) and is spaced a prescribed distance from the end at which the openings (1005) are located. That distance is great enough to enable the connector prong (1003) to unseat the ball (573) or other sealing device from the valve seat (501) before the shoulder (1007) bottoms out against the grommet (701), but is not great enough to enable the connector prong (1003) to drive the ball (573) against the cage (501) or permanently deform the deformable material (575). In other words, the shoulder (1007) encounters the upper surface of the grommet (701) before the ball (573) reaches the bottom of the cage (501). In this condition, most of the apertures (1005) in the connector prong (1003) are exposed to the cavity (503) in the cage (501) and the apertures (507) in the cage are not blocked by the ball (573). In an embodiment, the shoulder (1007) is formed from an elastomer so that it effects a seal with the surface of the grommet (701) around the outer end of the access bore (773) when in place. The distal end (1009) of the connector prong (1003) may be made of surgical grade steel or other material which is magnetic so as to attach and hold the ball (573) when the connector prong (1003) is in place. This type of arrangement can also facilitate returning the ball (573) to the valve seat (619) when the connector prong (1003) is withdrawn from the access passage.

Since the cavity (503) of the cage (501) is generally along the flow path of the fistula (100), apertures (507) in the cage (501) allow blood to be withdrawn from the patent's vascular system through the cannula (1001) to be cleansed. The treated blood is introduced back into the vascular system through another cannula (1001) which is inserted into another valve (300) located elsewhere in the body. Generally, both valves (300) will be located on the same fistula (100) in spaced relation to each other. Upon withdrawal of cannula (1001) after the procedure, a saline solution is preferably introduced through the cannula (1001) to cleanse the access bore (773) outwardly from the ball (573) and seat (763) as well as the cannula (1001). Likewise, pharmaceuticals may be introduced into the patient's vascular system through the cannula (1001) when in place in the valve (300).

When the valve element (573) or (575) is against the valve seat (619), blood flows through the lumen (201) of the fistula (100) without significant hindrance. Even if the cage (501) extends into the lumen (201), the apertures (507) will generally allow blood to flow through the cage (501) without major effect. If the cage (501) is above the lumen (such as in the embodiment of FIG. 2), there will be some surface effects due to the upper neck (307), but they would be relatively minor.

To implant the valve (300) in a patient requires a minor surgical procedure in which an incision is made in the patient's arm near where the fistula (100) has already been installed. The valve (300) is attached to the fistula (100) by any method known to one of ordinary skill in the art, based on the type of connecting member (303) present on the valve (300) and as discussed previously. The locking ring (801) is also placed through a hole in the skin and into the patient's arm. The grommet (701) is left outside the skin and the skin in closed to cover the locking ring (801). The grommet (701) is lowered onto the skin and the pins (711) penetrate the skin until they pass through the holes (803) and engage the ring (801) locking into place. One of ordinary skill in the art would also recognize that instead of installing valves (300) into a pre-existing fistula (100), a fistula (100) may be constructed or modified prior to installation to include one or more valves (300).

Once the valve (300) has been implanted in the patient's body, the valve (300) is available for introducing fluids into the patient's vascular system or withdrawing fluids from the system or both. As the surgery to install valves (300) in a pre-existing fistula (100) is significantly less invasive than the initial installation of the fistula (100) and there is no need to wait for the existence of sufficient grafting strength to existing vessels as such strength should already exist. The valve (300) may be available for immediate use upon installation. However, medical safety may indicate allowing additional healing before the valve (300) is used.

The valve (300) may be implanted in animals other than human beings but is of principle use in animals, including human animals, which require ready access to their blood and vascular system. Alternatively, the valve (300) may be used in other situations outside of access to blood and the vascular system. For instance, the valve (300) may be used to provide other types of medical or research access, such as to an airway, gastric system, or urinary tract. Further, the valve may be used outside the medical field to provide access to other fluid flow structures including but not limited to, pipes, fuel lines, exhaust lines or the like.

While the invention has been disclosed in connection with certain preferred embodiments, this should not be taken as a limitation to all of the provided details. Modifications and variations of the described embodiments may be made without departing from the spirit and scope of the invention, and other embodiments should be understood to be encompassed in the present disclosure as would be understood by those of ordinary skill in the art.

1. A valve comprising:
a connecting member for attaching said valve to a fistula;
a grommet, said grommet designed to be located on the exterior of the skin of a living animal, said grommet being connected to said connecting member;
an access passage, said access passage configured to project through the skin of said living animal, allowing fluid communication from a lumen in said fistula through said connecting member, said grommet, and to the exterior of said skin;
a valve element which normally bears against a sealing surface in said access passage to isolate said access passage from said lumen, but is displaceable away from said sealing surface such that the valve element and sealing surface are separated; thus enabling the access passage to be in fluid communication with said lumen.

2. A valve according to claim 1 wherein the sealing surface lies in a spherical envelope, and the valve element is spherical.

3. A valve according to claim 1 further comprising a cage which encloses a cavity which communicates with a lumen of said fistula; wherein the passage opens into the cavity, and the valve element is loosely received in the cavity.

4. A valve according to claim 1 wherein the valve element is biased toward the sealing surface.

5. A valve according to claim 4 also comprising a valve seat formed from a ferrous metal, with the sealing surface being on the valve seat and the passage extending through the valve seat; and wherein the valve element is magnetized.

6. A valve according to claim 4 wherein said valve element is biased via a spring.

7. A valve according to claim 1 wherein said valve element is a piece of deformable material.

8. A valve according to claim 1 wherein said grommet includes a plurality of locking pins, said locking pins allowing said grommet to be attached to a locking ring.

9. The valve of claim 8 wherein said skin of said living animal is held between said locking ring and said grommet.

10. The valve of claim 1 wherein said living animal is a human being.

11. The valve of claim 10 wherein said fistula is located in an arm of said human being.

12. The valve of claim 11 wherein said fistula is installed prior to the installation of said valve.
